(19) United States
(12) Patent Application Publication
(10) Pub. No.: US 2022/0400686 A1
(43) Pub. Date: Dec. 22, 2022

(54) COMPOSITIONS AND METHODS FOR ON-DEMAND RELEASE OF ANTIMICROBIAL AGENTS

(71) Applicant: University of Massachusetts, Boston, MA (US)

(72) Inventors: Jie Song, Shrewsbury, MA (US); Ananta Ghimire, Shrewsbury, MA (US)

(21) Appl. No.: 17/777,386

(22) PCT Filed: Nov. 24, 2020
(86) PCT No.: PCT/US20/61936

§ 371 Date: May 17, 2022

Related U.S. Application Data
(60) Provisional application No. 62/943,291, filed on Dec. 4, 2019.

(51) Int. Cl. A01N 63/50; A01N 25/10; A01P 1/00; A61L 27/54; A61L 27/52; A61L 27/06; A61L 27/34

(52) U.S. Cl. CPC A01N 63/50; A01N 25/10; A01P 1/00; A61L 27/54; A61L 27/52; A61L 27/06; A61L 27/34; A61L 2300/404

(57) ABSTRACT

The invention provides novel compounds and polymers, degradable hydrogel compositions, medical devices and implants, as well as methods thereof, that allow on-demand release and controlled delivery of antimicrobial agents.

### PRIORITY CLAIMS AND RELATED APPLICATIONS

This application claims the benefit of priority to U.S. Provisional Application No. 62/943,291, filed Dec. 4, 2019, the entire content of which is incorporated herein by reference for all purposes.

### STATEMENT REGARDING FEDERALLY FUNDED RESEARCH OR DEVELOPMENT

This invention was made with government support under grant numbers AR068418 and AR078044 awarded by the National Institutes of Health. The Government has certain rights in the invention.

### TECHNICAL FIELD OF THE INVENTION

The invention generally relates to hydrogels and delivery of therapeutics. More particularly, the invention relates to novel compounds and polymers, degradable hydrogel compositions, medical devices and implants, and methods of making and use thereof, that allow on-demand release and controlled delivery of antimicrobial (e.g., antibiotic) agents.

### BACKGROUND OF THE INVENTION

Periprosthetic infections represent occasional but serious health threats. Bacterial colonization around implants and subsequent biofilm formation are difficult to treat and could lead to implant failure, requiring major revision surgeries associated with high treatment cost, high morbidity and even mortality. There are no effective strategies for eradicating established biofilms or bacteria harboring within dense tissues such as the canaliculi of bone due to poor penetration of antibiotics and immune cells into the dense matrices of biofilm and cortical bone. (Wang, et al. 2017 Chemistry of Materials 29 (19), 8325-8337; Zhao, et al. 2009 J Biomed Mater Res B Appl Biomater 91 (1), 470-80; Campoccia, et al. 2006 Biomaterials 27 (11), 2331-9; Pichavant, et al. 2016 Biomacromolecules 17 (4), 1339-46; Cloutier, et al. 2015 Trends Biotechnol 33 (11), 637-652; Yu, et al. 2015 Acta Biomater 16, 1-13; de Mesy Bentley, et al. 2017 J Bone Miner Res 32 (5), 985-90.)

A current clinical practice for preventing infections in high-risk orthopedic joint replacement surgeries involves physical blending of antibiotics with bone cements. This approach, however, requires high antibiotic loading that could exert local and systemic cytotoxicity. Delivering antibiotics via non-covalent implant surface coating has been pursued as a safer alternative, although achieving suitable antibiotic release kinetics to ensure adequate and timely release remains a challenge. (Song, et al. 2016 ACS Appl Mater Interfaces 8 (22), 13785-92; Bakhshandeh, et al. 2017 ACS Appl Mater Interfaces 9 (31), 25691-25699; Aldrich, et al. 2019 ACS Appl Mater Interfaces 11 (13), 12298-12307; Zhuk, et al. 2014 ACS Nano 8, 8, 7733-7745. 2014; Anderson, et al. 2009 Biomaterials 30 (29), 5675-81; Stewart, et al. 2001 The Lancet 358 (9276), 135-138; Wang, et al. 2017 Biomacromolecules 19 (1), 85-93; Taubes 2008 Science 321, 356-61.)

Antibiotics covalently attached to implant surfaces have also been shown to exert bactericidal properties when they are presented via linkers or polymer chains of suitable flexibility/lengths at a modification site minimally perturbing the bioactivity of the drug. The limitation of this covalent surface modification approach, however, is that the antibiotic action is restricted to the immediate surface of the implant. (Pichavant, et al. 2016 Biomacromolecules 17 (4), 1339-46; Chen, et al. 2017 ACS Omega 2 (4), 1645-1652; Nie, et al. 2017 Antimicrob Agents Chemother 61 (1); Jose, et al. 2005 Chemistry & Biology 12 (9), 1041-1048; Lawson, et al. 2009 Biomacromolecules 10 (8), 2221-34; Walsh 2000 Nature 406 (6797), 775-81.)

Other approaches have been attempted including covalently conjugated vancomycin to the polymer brushes grafted from Ti6Al4V IM. Complete eradication of bacteria within the periprosthetic IM tissue environment, however, was not achieved due to the inability of the covalently tethered vancomycin to diffuse away from the Ti6Al4V surface. Additional challenges included cytotoxicity associated with the burst release of high doses of physically entrapped antibiotics or risks for developing bacteria resistance due to inadequate/delayed antibiotic releases. (Jose, et al. 2005 Chemistry & Biology 12 (9), 1041-1048; Lawson, et al. 2009 Biomacromolecules 10 (8), 2221-34; Walsh 2000 Nature 406 (6797), 775-81; Zhang, et al. 2019 ACS Appl Mater Interfaces. 11, 32, 28641-28647; Gerits, et al. 2016 J Orthop Res 34 (12), 2191-2198.)

More recently, delivery systems utilizing external stimuli such as pH, temperature, magnetic field, and ultrasound to trigger drug release were developed. For example, endogenous enzymatic activities (especially proteases and nucleases) have been exploited as more biologically relevant and safer alternatives to stimulate on-demand drug release from peptide- and nucleotide-based delivery systems. Non-specific cleavages of the peptide and oligonucleotide in these systems, however, present barriers to the success of such approaches. (Tang, et al. 2012 Advanced Materials 24 (12), 1504-1534; Li, et al. 2016 ACS Applied Materials & Interfaces 8 (3), 1842-1853; Zhou, et al. 2016 J Mater Chem B 4 (18), 3075-3085; Yang, et al. 1995 J Am Chem Soc 117 (17), 4843-4850; Yan, et al. 2006 J Am Chem Soc 128 (34), 11008-11009.) Oligonucleotide sequences with 2′-O-carboxymethyl modifications were recently shown to improve cleavage specificity by micrococcal nucleases (MN) of S. aureus. The therapeutic efficacy of the MN-triggered release of physically entrapped vancomycin in silica nanocapsules was demonstrated in vitro. However, in vivo efficacy of this approach, especially in the context of on-demand release of covalently tethered antibiotics for combating periprosthetic infections, has not been shown. (27. Yan, et al. 2006 J Am Chem Soc 128 (34), 11008-11009; Hernandez, et al. 2014 Nat Med 20 (3), 301-6; Hernandez, et al. 2014 Chem Commun (Camb) 50 (67), 9489-92.)

Preventing orthopedic implant associated bacterial infections remains a critical challenge. There is an urgent need for novel and improved strategies that ensure timely elimination of any bacteria present within the implant microenvironment, thereby preventing or minimizing biofilm formation or bacteria invasion.

### SUMMARY OF THE INVENTION

The invention provides a novel strategy that ensures timely elimination of any bacteria present within the implant or implantable tissue scaffold microenvironment, to prevent or reduce biofilm formation or bacteria invasion.

In one aspect, the invention generally relates to a polymer-drug conjugate comprising an oligonucleotide of about 2 to about 30 nucleotides in length having a first end and a second end, wherein the oligonucleotide is linked at the first end to a polymer and at the second end to an antimicrobial (e.g., antibiotic) agent.

In another aspect, the invention generally relates to a hydrogel composition comprising a polymer-drug conjugate disclosed herein.

In yet another aspect, the invention generally relates to a coating, surface or surface layer comprising a polymer-drug conjugate or a hydrogel composition disclosed herein.

In yet another aspect, the invention generally relates to a nanoparticle formulation comprising a polymer-drug conjugate or a hydrogel composition disclosed herein.

In yet another aspect, the invention generally relates to a medical device or implant comprising a polymer-drug conjugate, a hydrogel composition or a coating, surface or surface layer, or a nanoparticle formulation disclosed herein.

In yet another aspect, the invention generally relates to a medical device or implant comprising a coating, surface or a surface layer of a hydrogel composition, wherein the hydrogel composition comprises an oligonucleotide of about 2 to about 30 nucleotides in length having a first end and a second end, wherein the oligonucleotide is linked at the first end to a polymeric network and at the second end to an antibiotic agent, wherein the oligonucleotide is adapted to be cleaved by one or more microbial nucleases and not cleaved by a mammalian nuclease. The nucleotides flacking the potential cleavage site by specific bacterial enzymatic activities may include chemical modifications (e.g., 2′-O-carboxymethy and/or phosphorothioate modifications) for enhanced stability against non-specific enzymatic cleavages. The cleavage site can be located at varying number of base pairs from the drug end.

In yet another aspect, the invention generally relates to a compound having the structural formula:

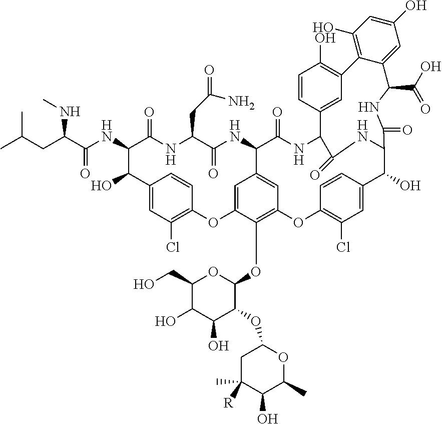

wherein R comprises an oligonucleotide having about 1 to about 29 nucleotide units.

In yet another aspect, the invention generally relates to a compound comprising an oligonucleotide of about 2 to about 30 nucleotides in length having a first end and a second end, wherein the oligonucleotide is linked at the first end to a first reactive group and at the second end to a second reactive group.

### BRIEF DESCRIPTION OF THE DRAWINGS

FIG. 1. Depiction of PEGDMA-Oligo-Vanco hydrogel network and MN-triggered vancomycin release. (a) Oligonucleotide (Oligo) sequence modified with bifunctional endgroups. (b) PEGDMA-Oligo hydrogel formation. (c) PEGDMA-Oligo-Vanco hydrogel formation and MN-triggered vancomycin release.

FIG. 2. MN-triggered oligo cleavage and the anti-bacterial activities of PEGDMA-Oligo-Vanco hydrogel in vitro. (a) GPC traces of intact oligo (black), oligo upon treatment with MN with (blue) and without (red) Ca²⁺. (b) Cumulative vancomycin (Vanco) release from PEGDMA-Oligo-Vanco hydrogel incubated with (black and blue) and without (red) MN. Differences at all given time points were significant (p≤0.0001). (c) Total bacterial counts after 24 h and 48 h of Xen-29 S. aureus culture in LB media containing PEGDMA-Oligo-Vanco, washed PEGDMA-Oligo/Vanco or PEGDMA-Oligo hydrogels (n=3; inset: corresponding IVIS image of the PEGDMA-Oligo-Vanco and PEGDMA-Oligo hydrogels retrieved after 48 h in S. aureus culture). (d) Photograph (left) and IVIS image (right) of an LB agar plate of Xen-29 S. aureus culture 24 h after placement of PEGDMA-Oligo-Vanco and PEGDMA-Oligo hydrogel discs (n=2 shown) over the agar plate. Error bars represent standard deviations. * p≤0.05, ** p≤0.01, *** p≤0.001, **** p≤0.0001 (two-way ANOVA).

FIG. 3. Surface modification and characterization of Ti6Al4V plates. (a) Schematic of sequential DopaMA and PEGDMA-Oligo hydrogel coatings on Ti6Al4V substrate. (b) Water contact angle (n=6) of Ti6Al4V and Ti6Al4V-DopaMA. Error bars represent standard deviations, **** p≤0.0001. (c) XPS scans on the Ti6Al4V surfaces before and after DopaMA immobilization. d) Dark field optical micrographs of PEGDMA-Oligo coating on Ti6Al4V-DopaMA vs. Ti6Al4V IM pins (1-mm in diameter). Magnification: 50×.

FIG. 4. Complete eradication of S. aureus inoculated in the mouse femoral canal by PEGDMA-Oligo-Vanco coating. (a) IVIS images of mouse femurs injected with 40 CFU Xen-29 S. aureus and inserted with IM pins with PEGDMA-Oligo-Vanco or PEGDMA-Oligo coatings at 2, 7, 14, and 21 days. (b) Quantification of longitudinal bioluminescence signals of mouse femurs injected with 40 CFU Xen-29 S. aureus and inserted with the different hydrogel-coated pins at 2, 7, 14, and 21 days (n=14). (c) S. aureus recovery from 21-day explanted pins (n=11). Error bars represent standard deviations. * p<0.05, ** p<0.01, *** p<0.001, **** p<0.0001 (two-way ANOVA for b; Student's t-test for c).

FIG. 5. Prevention of the development of osteomyelitis in mouse femoral canal inoculated with S. aureus by PEGDMA-Oligo-Vanco coating. (a) 3D μCT axial images of the distal femoral region 21 days after the insertion of Ti6Al4V IM pins (pins excluded during contouring) with different hydrogel coatings, with or without the inoculation of 40-CFU Xen-29 S. aureus; (b) Quantification of femoral BVF, BMD, and C. Th. of infected and uninfected femurs 21 days after the insertion of Ti6Al4V IM pins with PEGDMA-Oligo-Vanco or PEGDMA-Oligo coatings. n=11-14. Error bars represent standard deviations. * p≤0.05, ** p≤0.01 as compared to the PEGDMA-Oligo control coating+S. aureus group (one-way ANOVA). (c) H&E, AP (blue)/TRAP (red), and Gram staining of explanted femurs in the infected group with PEGDMA-Oligo-Vanco coating or PEGDMA-Oligo control coating at 21 days post-operation. Dashed lines outline the cortical bone; BM=bone marrow; BM*=infected bone marrow; Arrowheads indicate AP/TRAP activity; higher magnification views of the regions within the blue and red boxes are shown in the bottom row. Scale bars=500 μm (top and middle rows) or 100 μm (bottom row).

FIG. 6. Image of LB agar plate containing PEGDMA-Oligo-Vanco and PEGDMA-Oligo coated Ti6Al4V pins after 24 h S. aureus culture.

FIG. 7. In vivo study design.

FIG. 8. Longitudinal bioluminescence intensities of mouse femoral canals injected with 40 CFU Xen-29 S. aureus and inserted with IM pins with (n=14) or without (n=6) PEGDMA-Oligo-Vanco coating at 2, 7, 14, and 21 days. Error bars represent standard deviations. * p<0.05, ** p<0.01, ***p<0.001, ****p<0.0001 (two-way ANOVA).

FIG. 9. Post-op μCT axial images of the center slices of the mouse femurs confirming proper positioning of the inserted IM pins.

FIG. 10. (a) 3D reconstructed μCT images of the distal femoral region with the IM rod contoured out; (b) quantification of bone volume fraction (BVF), bone mineral density (BMD), and cortical thickness (C. Th.) of femurs in the PEGDMA-Oligo-Vanco+S. aureus (n=14) and unmodified Ti+S. aureus (n=6) groups at 21 days post-op. Error bars represent standard deviations.*p≤0.01, **p≤0.001 (student's t-test).

FIG. 11. H&E, AP/TRAP, and Gram staining of explanted femurs treated with PEGDMA-Oligo-Vanco coated pins with and without inoculated S. aureus, PEGDMA-Oligo coated pin without S. aureus, and unmodified Ti with S. aureus at 21 days post-operation. Dashed lines outline the cortical bone; BM=bone marrow; BM*=infected bone marrow; Arrowheads indicate AP/TRAP activity; Black box outlines Gram positive stained bacteria (red box showing zoomed-in view at 50× magnification); Scale bars=500 μm.

FIG. 12. H&E stained sections of heart, lung, liver, spleen, pancreas, kidney and rib retrieved from the mice receiving various IM pin treatments in uninfected vs. infected femoral canals for 21 days. Organs retrieved from age-matched mice without any treatment serve as normal controls (top row). 50× magnification. Scale bars=500 μm.

FIG. 13. Schematic depiction of covalent conjugation of an azide-terminated oligonucleotide in a hydrogel network by mixing it with azide- and cyclooctyne-terminated macromer building blocks with various fractions of hydrolysable ester linkages.

FIG. 14. GPC traces of intact oligos (black) and oligos after 0.5 h (for 0, 2 and 4 PS) and 24 h (for 6 PS) treatment with MN in the presence of Ca²⁺ (red), and blank (no oligo) control with MN and Ca′ only (blue) for (A) 0 PS oligo (B) 2 PS-a oligo (C) 4 PS oligo and (D) 6 PS oligo

FIG. 15. Image of LB agar plate of the S. aureus culture after the placement of PEGDMA-Oligo-Gentamicin and PEGDMA-Oligo-Tobramycin hydrogel discs for 24 h. Clear zones developed around the hydrogel discs indicate the bactericidal effect of the gentamicin or tobramycin released from the respective hydrogels due to cleavage of MN-sensitive oligo linkers with varying numbers of PS modifications by the S. aureus, with larger clear zones indicating more facile oligo linker cleavage. PEGDMA-Oligo hydrogels with physically encapsulated but subsequently washed away antibiotics served as negative controls.

FIG. 16. Total bacterial counts after 24-h culture of Xen-29 S. aureus in the human serum with/without MN recovered after incubating with PEGDMA-Oligo-Gentamicin hydrogel for 2 h (n=3).

### DETAILED DESCRIPTION OF THE INVENTION

The invention provides novel compositions and methods for timely elimination of any bacteria from the implant microenvironment and prevention of biofilm formation or bacteria invasion. Covalent attachment of antibiotics to the implant surface via a linker sensitive to unique bacterial enzymatic activities was shown to timely release free antibiotics to combat infections within a broader periprosthetic tissue microenvironment.

As demonstrated by a proof-of concept example disclosed herein, covalently functionalized poly(ethylene glycol) dimethacrylate hydrogel coating having vancomycin conjugated via an oligonucleotide linker sensitive to micrococcal nuclease (MN) (PEGDMA-Oligo-Vanco) was shown to timely release vancomycin in the presence of MN or Staphylococcus aureus (S. aureus), the gram-positive bacterium responsible for a third of all orthopedic implant related infections and a major cause for osteomyelitis, and eradicate the bacteria both from the implant surface and within the marrow cavity and preventing their invasion to the cortical bone and subsequent development of osteomyelitis. Ti6Al4V intramedullary (IM) pins surface-tethered with dopamine methacrylamide (DopaMA) and uniformly coated with PEGDMA-Oligo-Vanco effectively prevented periprosthetic infections in mouse femoral canals inoculated with bioluminescent S. aureus. Longitudinal bioluminescence monitoring, μCT quantification of femoral bone changes, endpoint quantification of implant surface bacteria and histological detection of S. aureus in the periprosthetic tissue environment confirmed rapid and sustained bacterial clearance by the PEGDMA-oligo-Vanco coating. The observed eradication of bacteria was in stark contrast with the significant bacterial colonization on implants and osteomyelitis developed in the absence of the MN-sensitive bactericidal coating. The effective vancomycin tethering dose presented in this on-demand release strategy was >200 times lower than the typical prophylactic antibiotic contents used in bone cements and can be readily applied to medical implants and bone/dental cements to prevent periprosthetic infections in high-risk clinical scenarios. This disclosure also supports the timely bactericidal action by MN-triggered release of antibiotics as an effective prophylactic method to bypass the notoriously harder to treat periprosthetic biofilms and osteomyelitis.

The PEG-based polymethacrylate gelling mechanism was shown to be compatible with the solidification of bone cement and dental resin, thus can be readily applied as prophylactic standard care to prevent implant-associated biofilm formation and osteomyelitis. The DopaMA intermediate coating applied to the metallic implant surfaces ensures the stable and uniform hydrogel coating on the metallic implant surface. Importantly, the low effective antibiotic tethering dose (e.g., more than 2 orders of magnitude reduction compared to prophylactic antibiotics physically blended with bone cement) and the MN-sensitive on-demand release mechanism improve both the efficacy (timely release) and safety of local antibiotics delivery.

In one aspect, the invention generally relates to a polymer-drug conjugate comprising an oligonucleotide of about 2 to about 30 (e.g., about 2 to about 26, about 2 to about 22, about 2 to about 18, about 2 to about 14, about 2 to about 12, about 2 to about 8, about 2 to about 4, about 4 to about 30, about 6 to about 30, about 8 to about 30, about 12 to about 30, about 16 to about 30, about 4 to about 20, about 6 to about 16) nucleotides in length having a first end and a second end, wherein the oligonucleotide is linked at the first end to a polymer and at the second end to an antimicrobial (e.g., antibiotic) agent.

The nucleotides flacking the cleavage site by specific bacterial enzymatic activities may include chemical modifications (e.g., 2′-O-carboxymethy and/or phosphorothioate modifications) for enhanced stability against non-specific enzymatic cleavages. The cleavage site can be located at varying number of base pairs from the drug end.

In certain embodiments, the oligonucleotide is a single stranded oligonucleotide.

In certain embodiments, the oligonucleotide is a fusion of one or more (e.g., two or more) single or double stranded oligonucleotides.

In certain embodiments, the oligonucleotide is adapted to be selectively cleaved by a first microbial nuclease. In certain embodiments, the microbial nuclease is micrococcal nucleases (MN) of S. aureus.

In certain embodiments, the oligonucleotide is stable against mammalian nucleases.

In certain embodiments, the oligonucleotide is adapted to be cleaved by a second or further microbial nucleases.

In certain embodiments, the oligonucleotide is about 2 to about 20 nucleotides in length.

In certain embodiments, the oligonucleotide comprises a sequence selected from the group consisting of XabY, wherein X and Y are any combination of no more than 28 of A, fA, A*, mA, mA*, fA*, C, fC, C*, mC, mC*, fC*, G, fG, G*, mG, mG*, fG*, U, fU, U*, mU, mU*, fU*, T, T*, wherein f denotes 2′-fluoro modification, m denotes 2′-O-methylation while * denotes phosphorothioate modification, and a and b are unmodified nucleotides (identical or different). For example, XabY may be mC-mG-T-T-mC-mG, C*-G*-T-T-C*-G*, mC*-mG*-T-T-mC*-mG*, mC-mG*-T-T-mC*-mG, mC*-mG-T-T-mC-mG*, mC-mU-mC-mG-T-T-mC-mU-mC-mG, etc. (See, also, US20190247507A1 and US20180179546A1)

In certain embodiments, the oligonucleotide comprises one or more chemically modified pyrimidines and purines.

In certain embodiments, the one or more chemical modifications comprise one or more of 2′-fluoro modification, 2′-O-carboxymethyl modifications and/or one or more phosphorothioate modifications.

In certain embodiments, a first spacer is present between the oligonucleotide and the polymer and a second spacer between the oligonucleotide and the antimicrobial agent.

In certain embodiments, the first and/or second spacers is a hydrophobic group (e.g. a hydrocarbon). In certain embodiments, the first and/or second spacers is a hydrophilic group (e.g., comprising ethylene glycol unit(s)). The first and/or second spacers may be any suitable length, e.g., having about 1 to about 10 (e.g., 1, 2, 3, 4, 5, 6, 7, 8, 9, 10) carbon atoms and 0 to 5 (e.g., 0, 1, 2) heteroatoms (e.g., O, S or N).

In certain embodiments, each of the first and second spacers is independently —(CH₂)ᵢ—, wherein i is an integer from about 1 to about 10 (e.g., 1, 2, 3, 4, 5, 6, 7, 8, 9, 10).

In certain embodiments, the polymer comprises a monomer comprising one or more ethylene glycol units. In certain embodiments, the polymer comprises a monomer comprising dimethacrylate.

In certain embodiments, the polymer comprises a monomer of poly(ethylene glycol) dimethacrylate.

In certain embodiments, antimicrobial agent is selected from vancomycin, gentamicin, and tobramycin. In certain embodiments, antimicrobial agent is vancomycin. In certain embodiments, antimicrobial agent is gentamicin. In certain embodiments, antimicrobial agent is tobramycin.

In certain embodiments, antimicrobial agent is selected from and cephalosporins, quinolones. In certain embodiments, antimicrobial agent is a cephalosporin. In certain embodiments, antimicrobial agent is a quinolone. In certain embodiments, the cephalosporin or quinolone comprises a primary amine, alcohol or carboxylate group.

In certain embodiments, the polymer is covalently linked to a surface of an implant.

In certain embodiments, the implant is a metallic implant. In certain embodiments, the implant is selected from the group consisting of implantable synthetic tissue scaffolds.

In certain embodiments, the implant is selected from the group consisting of a catheter, a vascular stent, a dental implant, and an orthopedic implant.

In certain embodiments, the covalent linkage to the implant surface is via metal oxide-binding chemical functionalities.

In another aspect, the invention generally relates to a hydrogel, degradable or non-degradable, composition comprising a polymer-drug conjugate disclosed herein.

In yet another aspect, the invention generally relates to a coating, surface or surface layer comprising a polymer-drug conjugate or a hydrogel composition disclosed herein.

In yet another aspect, the invention generally relates to a medical device or implant comprising a polymer-drug conjugate, a hydrogel composition or a coating, surface or surface layer disclosed herein.

In yet another aspect, the invention generally relates to a medical device or implant comprising a coating, surface or a surface layer of a hydrogel composition, wherein the hydrogel composition comprises an oligonucleotide of about 2 to about 30 (e.g., about 2 to about 26, about 2 to about 22, about 2 to about 18, about 2 to about 14, about 2 to about 12, about 2 to about 8, about 2 to about 4, about 4 to about 30, about 6 to about 30, about 8 to about 30, about 12 to about 30, about 16 to about 30, about 4 to about 20, about 6 to about 16) nucleotides in length having a first end and a second end, wherein the oligonucleotide is linked at the first end to a polymeric network and at the second end to an antibiotic agent, wherein the oligonucleotide is adapted to be selectively cleaved by a first microbial nuclease and resistant to cleavage (e.g., substantially more stable against cleavage) by a mammalian nuclease.

In certain embodiments of the medical device or implant, the hydrogel is degradable. In certain embodiments, the hydrogel is non-degradable.

In certain embodiments, the medical device or implant is characterized by having a metallic surface.

In certain embodiments of the medical device or implant, the metallic surface comprises Ti6Al4V.

In certain embodiments of the medical device or implant, the oligonucleotide is single stranded.

In certain embodiments of the medical device or implant, the oligonucleotide is a fusion of one or more (e.g., two or more) single or double stranded oligonucleotides.

In certain embodiments of the medical device or implant, the oligonucleotide is about 4 to about 20 nucleotides in length.

In certain embodiments of the medical device or implant, a first spacer is present between the oligonucleotide and the polymeric network and a second spacer between the oligonucleotide and the antibiotic agent.

In certain embodiments, the first and/or second spacers is a hydrophobic group (e.g. a hydrocarbon). In certain embodiments, the first and/or second spacers is a hydrophilic group (e.g., comprising ethylene glycol unit(s)). The first and/or second spacers may be any suitable length, e.g., having about 1 to about 10 (e.g., 1, 2, 3, 4, 5, 6, 7, 8, 9, 10) carbon atoms and 0 to 5 (e.g., 0, 1, 2) heteroatoms (e.g., O, S or N).

In certain embodiments of the medical device or implant, each of the first and second spacers is independently —(CH₂)ᵢ—, wherein i is an integer from about 1 to about 10 (e.g., 1, 2, 3, 4, 5, 6, 7, 8, 9, 10).

The nucleotides flacking the cleavage site by specific bacterial enzymatic activities may include chemical modifications (e.g., 2′-O-carboxymethy and/or phosphorothioate modifications) for enhanced stability against non-specific enzymatic cleavages. The cleavage site can be located at varying number of base pairs from the drug end.

In certain embodiments of the medical device or implant, the oligonucleotide comprises one or more chemically modified pyrimidines and purines.

In certain embodiments of the medical device or implant, the one or more chemical modifications comprise one or more of 2′-fluoro modification, 2′-O-carboxymethyl modifications and/or one or more phosphorothioate modifications.

In certain embodiments of the medical device or implant, the oligonucleotide is single stranded. In certain embodiments of the medical device or implant, the oligonucleotide is double stranded.

In certain embodiments, the oligonucleotide comprises a sequence selected from the group consisting of XabY, wherein X and Y are any combination of no more than 28 of A, fA, A*, mA, mA*, fA*, C, fC, C*, mC, mC*, fC*, G, fG, G*, mG, mG*, fG*, U, fU, U*, mU, mU*, fU*, T, T*, wherein f denotes 2′-fluoro modification, m denotes 2′-O-methylation while * denotes phosphorothioate modification, and a and b are unmodified nucleotides (identical or different). For example, XabY may be mC-mG-T-T-mC-mG, C*-G*-T-T-C*-G*, mC*-mG*-T-T-mC*-mG*, mC-mG*-T-T-mC*-mG, mC*-mG-T-T-mC-mG*, mC-mU-mC-mG-T-T-mC-mU-mC-mG, etc. (See, also, US20190247507A1 and US20180179546A1)

In certain embodiments, the oligonucleotide comprises one or more chemically modified pyrimidines and purines.

In yet another aspect, the invention generally relates to a compound having the structural formula:

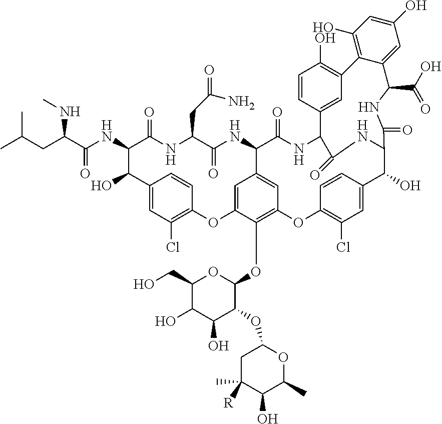

wherein R comprises an oligonucleotide having about 1 to about 29 (e.g., about 1 to about 29, about 3 to about 29, about 5 to about 29, about 9 to about 29, about 1 to about 23, about 1 to about 19, about 1 to about 15, about 1 to about 11, about 1 to about 7, about 1 to about 3, about 2 to about 9, about 3 to about 7) nucleotide units.

In certain embodiments of the compound, R comprises —NH—C(═O)O—.

In certain embodiments of the compound, the oligonucleotide has about 2 to about 19 nucleotide units.

In certain embodiments of the compound, R comprises a spacer between the oligonucleotide and the remaining of the compound.

In certain embodiments, the spacer is a hydrophobic group (e.g. a hydrocarbon). In certain embodiments, the spacer is a hydrophilic group (e.g., comprising ethylene glycol unit(s)). The spacer may be any suitable length, e.g., having about 1 to about 10 (e.g., 1, 2, 3, 4, 5, 6, 7, 8, 9, 10) carbon atoms and 0 to 5 (e.g., 0, 1, 2) heteroatoms (e.g., O, S or N).

In certain embodiments of the compound, the spacer is —(CH₂)ᵢ—, wherein i is an integer from about 1 to about 10 (e.g., 1, 2, 3, 4, 5, 6, 7, 8, 9, 10).

In yet another aspect, the invention generally relates to a compound comprising an oligonucleotide of about 2 to about 30 (e.g., about 2 to about 26, about 2 to about 22, about 2 to about 18, about 2 to about 14, about 2 to about 12, about 2 to about 8, about 2 to about 4, about 4 to about 30, about 6 to about 30, about 8 to about 30, about 12 to about 30, about 16 to about 30, about 4 to about 20, about 6 to about 16) nucleotides in length having a first end and a second end, wherein the oligonucleotide is linked at the first end to a first reactive group and at the second end to a second reactive group.

In certain embodiments, the oligonucleotide is adapted to be cleaved by a microbial nuclease. In certain embodiments, the microbial nuclease is micrococcal nucleases (MN) of S. aureus.

In certain embodiments, the oligonucleotide is not cleaved by a mammalian nuclease.

In certain embodiments, a first spacer is present between the oligonucleotide and the first reactive group and a second spacer between the oligonucleotide and the second reactive group.

In certain embodiments, the first and/or second spacers is a hydrophobic group (e.g. a hydrocarbon). In certain embodiments, the first and/or second spacers is a hydrophilic group (e.g., comprising ethylene glycol unit(s)). The first and/or second spacers may be any suitable length, e.g., having about 1 to about 10 (e.g., 1, 2, 3, 4, 5, 6, 7, 8, 9, 10) carbon atoms and 0 to 5 (e.g., 0, 1, 2) heteroatoms (e.g., O, S or N).

In certain embodiments, each of the first and second spacers is independently —(CH₂)ᵢ—, wherein i is an integer from about 1 to about 10 (e.g., 1, 2, 3, 4, 5, 6, 7, 8, 9, 10).

In certain embodiments, the oligonucleotide comprises one or more chemically modified pyrimidines and purines.

In certain embodiments, the one or more chemical modification comprises one or more of 2′-O-carboxymethyl modifications.

In certain embodiments, the first reactive group is a methacryl group. In certain embodiments, the first reactive group is an azide.

In certain embodiments, the second reactive group is a carboxylic acid group. In certain embodiments, the second reactive group is an amine group.

In certain embodiments, the oligonucleotide is single stranded.

In certain embodiments, the oligonucleotide comprises a sequence selected from the group consisting of XabY, wherein X and Y are any combination of no more than 28 of A, fA, A*, mA, mA*, fA*, C, fC, C*, mC, mC*, fC*, G, fG, G*, mG, mG*, fG*, U, fU, U*, mU, mU*, fU*, T, T*, wherein f denotes 2′-fluoro modification, m denotes 2′-O-methylation while * denotes phosphorothioate modification, and a and b are unmodified nucleotides (identical or different). For example, XabY may be mC-mG-T-T-mC-mG, C*-G*-T-T-C*-G*, mC*-mG*-T-T-mC*-mG*, mC-mG*-T-T-mC*-mG, mC*-mG-T-T-mC-mG*, mC-mU-mC-mG-T-T-mC-mU-mC-mG, etc. (See, also, US20190247507A1 and US20180179546A1)

In certain embodiments, the compound is covalently conjugated at the first end to a polymer.

In certain embodiments, the compound is covalently conjugated at the second end to an antibiotic agent.

In certain embodiments, the compound is covalently conjugated at the first end to a polymer and at the second end to an antibiotic agent.

The following examples are meant to be illustrative of the practice of the invention, and not limiting in any way.

### Examples

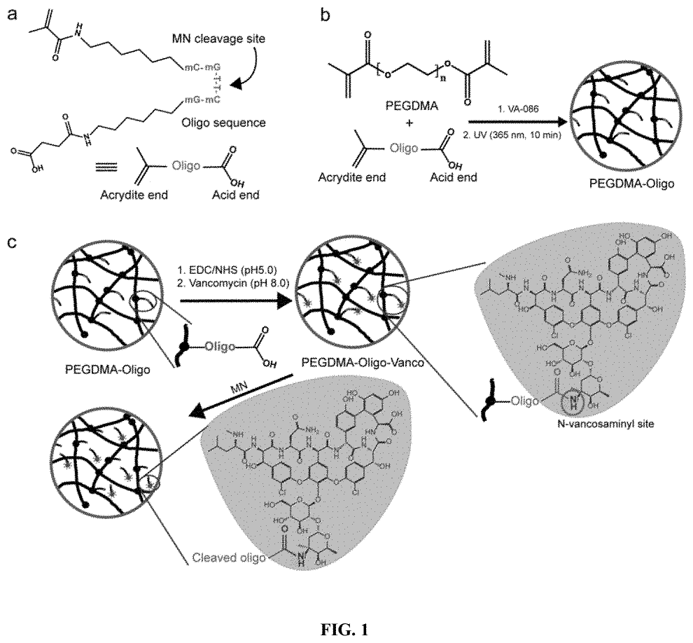

As disclosed herein, covalently functionalize PEGDMA hydrogel with vancomycin via a 2′-O-carboxymethyl modified oligonucleotide linker sensitive to S. aureus MN was synthesized and evaluated its antibacterial activity in vitro and anti-periprosthetic infection properties in vivo upon application to metallic implants as a surface coating using a rodent femoral canal infection model. The oligonucleotide linker (abbreviated as oligo, FIG. 1a) included a carboxylic acid and an acrydite on either end of the mC-mG-T-T-mC-mG sequence. The sequence was previously shown to exhibit enhanced stability to mouse and human serum but sensitivity to S. aureus MN cleavages. (Hernandez, et al. 2014 Chem Commun (Camb) 50 (67), 9489-92; Hernandez, et al. 2014 Nature Medicine 20 (3), 301-306.)

The acrydite end was employed to covalently conjugate the probe to the PEGDMA matrix during radical polymerization (FIG. 1B) while the carboxylic acid end of the oligo linker was used to form amide linkage with the N-vancosaminyl group of vancomycin via EDC/NHS chemistry (FIG. 1c).

Vancomycin, a glycopeptide antibiotic acting at the Gram-positive bacterial cell walls to block peptidoglycan synthesis, is considered the most effective in treating infections caused by Staphylococcus including Methicillin-resistant S. aureus. (Grundmann, et al. 2006 Lancet 368 (9538), 874-85.) It inhibits the transpeptidation and transglycosylation steps of bacterial cell wall biosynthesis through the binding of the L-Lys-D-Ala-D-Ala termini of the nascent peptidoglycan precursor through H-bonds. Chemical modification at the N-vancosaminyl site is known to present minimal perturbation to this binding. (Kahne, et al. 2005 Chem Rev 105 (2), 425-48; Lawson, et al. 2009 Biomacromolecules 10 (8), 2221-2234.)

The flexible C5 spacers between the nucleotide sequence and the bifunctional endgroups were designed to relieve steric hindrance during amidation with vancomycin via the N-vancosaminyl site and to ensure adequate rotational freedom of the oligo upon covalent attachment to the hydrogel matrix. In the presence of metabolically active S. aureus, the bacterial MN is expected to cleave the oligo at the unmodified T-T position, releasing the vancomycin with a small overhung fragment at the N-vancosaminyl site (FIG. 1c). As previously shown, a minimal effective concentration of vancomycin against S. aureus was not significantly altered upon modification with an N-vancosaminyl oligo(ethylene glycol) overhang, which is of comparable length of the amidated oligo fragment overhang upon MN-cleavage. (Zhang, et al. 2019 ACS Appl Mater Interfaces. 11, 32, 28641-28647.)

### Synthesis of PEGDMA-Oligo and PEGDMA-Oligo-Vanco Hydrogels

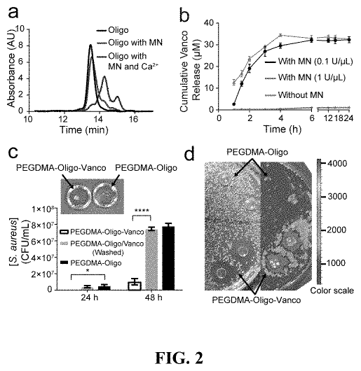

The cleavage of the oligo probe (synthesized and purified by Integrated DNA Technologies) by MN (source: S. aureus strain ATCC #27735; Worthington Biochemical Corporation) was first validated prior to its conjugation with vancomycin or PEGDMA hydrogel. Gel permeation chromatography (GPC) and UV detection at 260 nm revealed two fragments upon treatment of the oligo by MN (0.1 U/μL) in PBS (pH 7.4, 10 mM) containing a physiological concentration of calcium ions (FIG. 2a, blue trace). In the absence of calcium ions, MN showed very little cleavage of oligo (FIG. 2a, red trace).

Upon confirmation of the sensitivity of the oligo to MN cleavage, it was covalently incorporated with PEGDMA during photo-polymerization. A leaching experiment showed that 90±5.0% oligo was effectively tethered to a 15 w/v % PEGDMA hydrogel, achieving 1.1-μg oligo/mg PEGDMA incorporation content, as revealed by the detection of untethered oligo (absorption at 260 nm) leached into DI water upon extensive equilibration of the PEGDMA-Oligo hydrogel in PBS (48 h).

Covalent attachment of vancomycin to PEGDMA-Oligo hydrogel was carried out using EDC/NHS chemistry resulting in the PEGDMA-Oligo-Vanco hydrogel. To examine the release of covalently tethered vancomycin from the hydrogel, PEGDMA-Oligo-Vanco hydrogel discs (50 μL, 8 mm in diameter, n=3) were incubated in PBS (pH 7.4, 10 mM) containing a physiological concentration of calcium ions with or without the presence of MN (0.1 and 1.0 U/μL). The concentration of released vancomycin was monitored by UV spectroscopy at 280 nm. As shown in FIG. 2b, the release of vancomycin was only detected in the group spiked with MN, supporting cleavage of the oligo linker and release of vancomycin by MN activity. The initial release (first 4 h) of vancomycin was slightly higher with the higher MN concentration, although the total vancomycin released over 24 h was the same. Assuming complete release of vancomycin after 24 h, the amount of vancomycin loaded on the 15 w/v % PEGDMA hydrogel matrix was 90-μg/mm³ hydrogel (or 24-mg Vanco/40-g PEGDMA; note that the minimum inhibitory concentration of vancomycin was μg/mL). This vancomycin covalent loading content was significantly lower than the non-covalent prophylactic antibiotics incorporation content in commercial bone cement (e.g., 0.5-1.0-g gentamycin/40-g bone cement, 1.0-g tobramycin/40-g bone cement). (Bistolfi, et al. 2011 ISBN Orthop 2011, 290851; Neut, et al. 2005 J Biomed Mater Res A 73 (2), 165-70; Meyer, et al. 2011 J Bone Joint SurgAm 93 (22), 2049-56; Jiranek, et al. 2006 J Bone Joint SurgAm 88 (11), 2487-500.)

### In Vitro Antibacterial Activity of PEGDMA-Oligo-Vanco Hydrogel

The therapeutic efficacy of PEGDMA-Oligo-Vanco hydrogel was first evaluated by in vitro bacterial cultures. PEGDMA-Oligo-Vanco and PEGDMA-Oligo hydrogel discs (50 μL, 8 mm in diameter, n=3) were incubated in LB media containing 130 CFU of S. aureus at 37° C. To account for potential contribution from residue unconjugated vancomycin, another control group with the same content of vancomycin physically entrapped (without EDC/NHS) at the time of PEGDMA-Oligo hydrogel formation was also prepared (PEGDMA-Oligo/Vanco). All hydrogels were washed for 72 h in DI water before being placed in Xen-29 S. aureus suspension culture. The Xen29 S. aureus, emitting bioluminescence when metabolically active due to the expression of luciferase³⁸, enables convenient visualization in vitro and longitudinal monitoring in vivo by an in vivo imaging system (IVIS-100, Perkin-Elmer).

As shown in FIG. 2c, the PEGDMA-Oligo-Vanco hydrogel was able to significantly inhibit the bacterial growth by 8-fold after 48 h. Of note, IVIS imaging showed no signs of bacterial attachment and colonization on the surface of the PEGDMA-Oligo-Vanco hydrogel upon its retrieval from the suspension culture after 48 h whereas significant colonization of S. aureus was detected on the PEGDMA-Oligo control hydrogel (FIG. 2c inset). The cleavage of the oligo linker by live S. aureus and the ability of released vancomycin to diffuse out of the hydrogel to exert antibiotic activities were further validated by the clear zone development around the PEGDMA-Oligo-Vanco hydrogels placed on an agar plate of the S. aureus culture. No clear zone was developed around the PEGDMA-Oligo control. These in vitro outcomes indicate that when applied to the surface of implants, the PEGDMA-Oligo-Vanco hydrogel coating may also inhibit S. aureus surface attachment and colonization and suppress or even eradicate bacterial growth in its vicinity.

### Hydrogel Coating of Ti6Al4V IM Pins

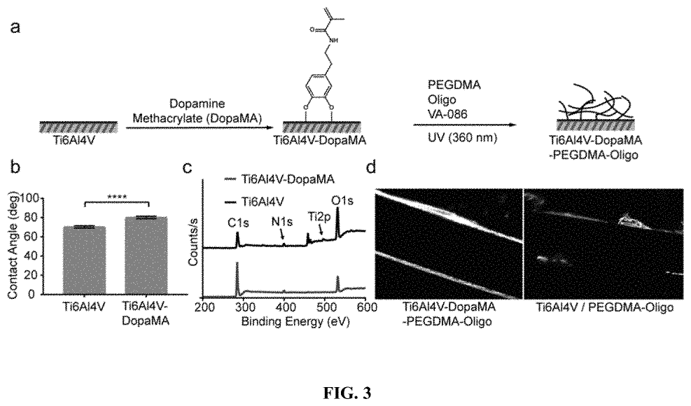

To evaluate the ability of PEGDMA-Oligo-Vanco hydrogel coating to prevent or mitigate periprosthetic infections in vivo, the coating was applied to the surface of Ti6Al4V IM pins emulating the metallic hardware used in orthopedic surgeries. To enhance the adhesion and stability of the hydrogel coating to the metallic substrate, the Ti6Al4V surface was first treated with dopamine methacrylate (DopaMA, FIG. 3a). The catechol group from DopaMA is known for high affinity for surface oxides of Ti6Al4V (Chai, et al. 2019 ACS Biomater. Sci. Eng. 5, 6, 2708-2724.) whereas the methacrylate is designed to covalently polymerize with the PEGDMA matrix during hydrogel coating application. Dip-coating the metallic substrate with DopaMA was used to promote a more uniform and stable surface coating of the functionalized PEGDMA. The choice of the methacrylate surface group also makes it possible to covalently bond with poly(methyl methacrylate) (PMMA) bone cement if desired. Successful surface modification of Ti6Al4V plates (1 cm×cm) with DopaMA was confirmed by water contact angle measurements and X-ray Photoelectron Spectroscopy (XPS) analyses.

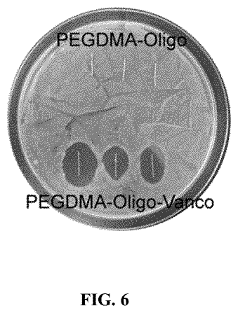

A statistically significant increase in water contact angle (FIG. 3b) was observed upon DopaMA surface coating, consistent with the increased surface hydrophobicity due to the bonding of the more hydrophilic catechol unit with surface oxides, exposing the hydrophobic methacrylate towards the air. Decreases in XPS signal intensities for Ti2p and O1s and an increase in intensity for C1s were also observed upon DopaMA treatment, consistent with the surface coverage by the organic molecules (FIG. 3c). Hydrogel precursors for PEGDMA-Oligo were then photo polymerized onto the DopaMA-coated Ti6Al4V surface. As shown by dark-field optical microscopy (FIG. 3d), the DopaMA intermediate improved the uniformity of the PEGDMA-Oligo coating on the Ti6Al4V pins (0.5-mm in diameter; to be inserted into mouse femoral canal in subsequent in vivo studies). Vancomycin was then covalently attached to the hydrogel coating by EDC/NHS chemistry as described earlier. The antibacterial capability of PEGDMA-Oligo-Vanco-coated Ti6Al4V pins were validated by in vitro bacterial culture on LB agar plates (FIG. 6).

In Vivo Antibacterial Activity of Ti6Al4V Pins Coated with PEGDMA-Oligo-Vanco Hydrogel

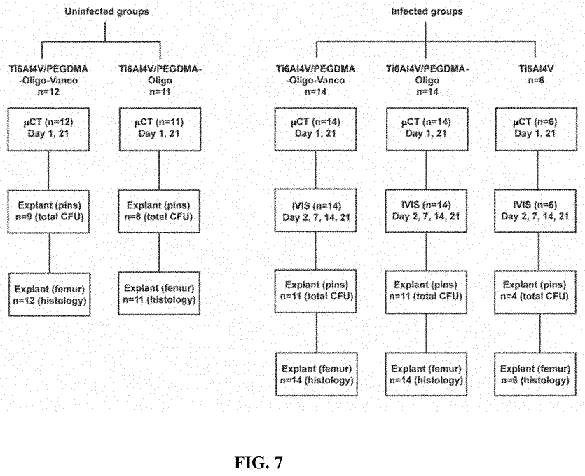

A rodent femoral canal infection model was used to investigate the antibacterial activity of the hydrogel-coated Ti6Al4V IM pins in vivo, a mouse femoral canal infection model was used. (Antoci, et al. 2007 Clin Orthop Relat Res 461, 88-95; Francis, et al. 2000 Infect Immun 68 (6), 3594-600.) A low dose (40 CFU) of bioluminescent Xen29 S. aureus was inoculated in the reamed femoral canal of skeletally mature CL57BL/6 mice (6-10 weeks old, males) before an unmodified Ti6Al4V pin, or a Ti6Al4V pin coated with PEGDMA-Oligo-Vanco or PEGDMA-Oligo hydrogel was inserted (FIG. 7). This low bacteria inoculation dose was chosen to emulate a realistic clinical scenario where gross contamination during arthroplasty surgery is rare. The degree of infection was evaluated by femoral cortical bone thickening and bone mineral density changes by longitudinal micro computed tomography (μCT), quantification of total bacterial counts on retrieved pins, and detection of S. aureus within explanted femur by Gram staining at the 21-day endpoint.

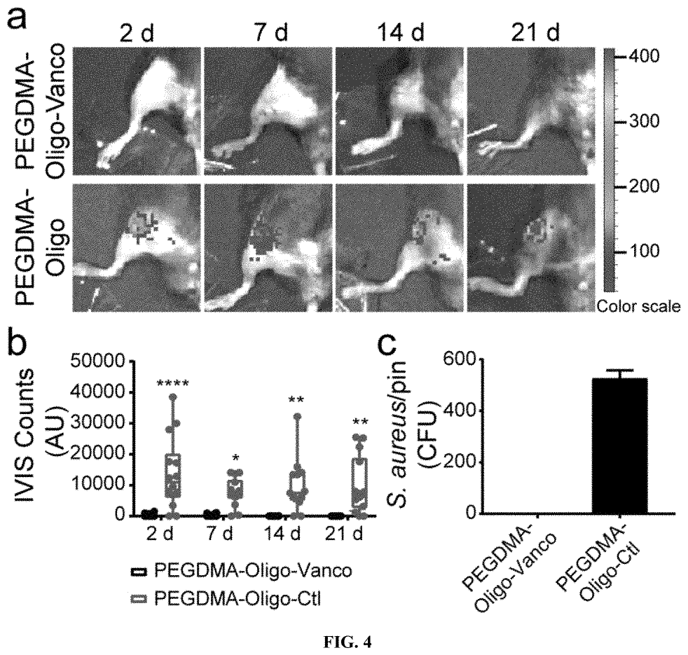

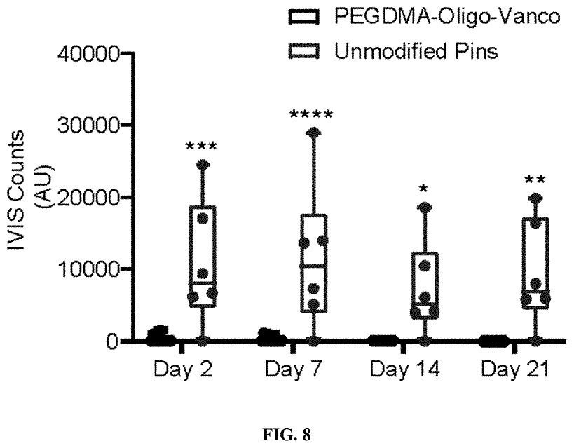

IVIS detected bioluminescence at the femurs receiving the IM pins coated with PEGDMA-Oligo control hydrogel at 2 days post-operation and the bioluminescence sustained over the course of 21 days (FIG. 4a, bottom panel; FIG. 4b, red bars). The longitudinal detection of bioluminescence in the no-vancomycin control coating group further validated the establishment of infection with the inoculation of 40 CFU Xen29 S. aureus. By stark contrast, no obvious bioluminescence was visualized from the femurs inserted with the IM pins coated with PEGDMA-Oligo-Vanco at any timepoint during the 21-day follow-up (FIG. 4a, bottom panel), and the quantification of bioluminescent signals confirmed significant reduction in intensity by >95% at 2 days post-operation compared to the control groups (pins with PEGDMA-Oligo coating, FIG. 4b, black bars; or unmodified pins, FIG. 8), and complete disappearance after day 7 (FIG. 4b, red bars). To confirm the elimination of bacterial burden by the PEGDMA-Oligo-Vanco coating in vivo, the IM pins were harvested on day 21 and thoroughly vortexed (5 min in LB media) before the suspensions were cultured on LB agar plates for bacterial counts. Consistent with the IVIS imaging data, no bacteria were recovered from the retrieved IM pins with PEGDMA-Oligo-Vanco coating, supporting complete eradication of peri-prosthetically bound bacteria while >500 CFU S. aureus were recovered from the retrieved IM pins with the PEGDMA-Oligo control coating (FIG. 4c).

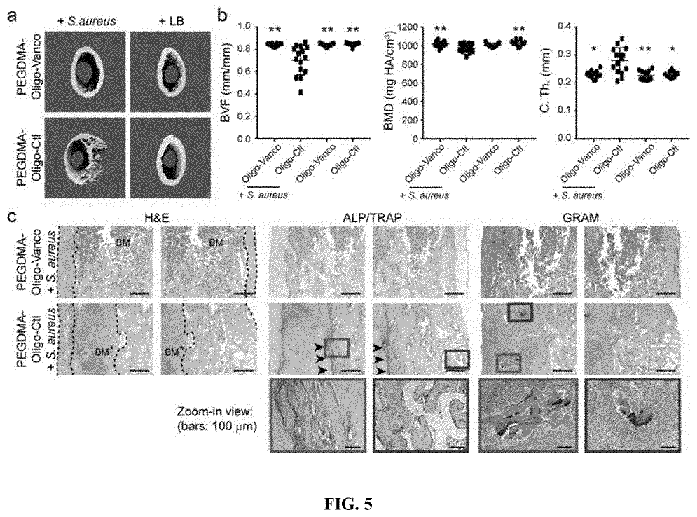

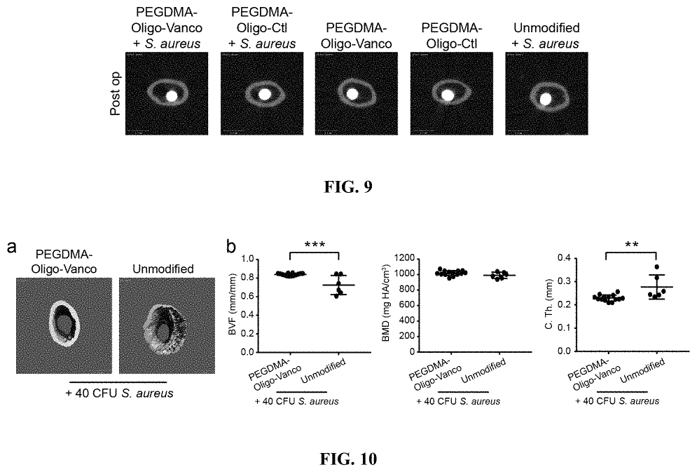

μCT imaging was carried out post-operation to confirm the proper positioning of inserted pins in all groups (FIG. 9) and at the 3-week endpoint to determine the degree of infection within the femoral region of interest (ROI). Established local infections (osteomyelitis) could deteriorate the bone quality over time, causing decreases in bone volume fraction (BVF) (Stadelmann, et al. 2015 Biomed Res Int 2015, 587857) and bone mineral density (BMD) (Niska, et al. 2012 PLoS One 7 (10), e47397), and resulting in cortical thickening (Odekerken, et al. 2014 Biomed Res Int 2014, 424652.) in the affected area. Accordingly, these properties were quantified by endpoint μCT in both infected and uninfected femurs treated with IM pins with or without hydrogel coatings. 3D μCT axial slice images at the endpoint. 3D μCT axial slice images at endpoint revealed osteolysis and an increase in cortical thickness (C. Th.), most clearly observed in the distal region, of the infected femurs treated with unmodified pins (FIG. 10a) or pins coated with PEGDMA-Oligo control hydrogel (FIG. 5a). These changes were confirmed by the significant decreases in BVF and BMD accompanying the increase in C. Th. in the PEGDMA-Oligo+S. aureus group (FIG. 5b) and the unmodified pin+S. aureus group (FIG. 10b) compared to the uninfected groups or infected group treated with pins coated with PEGDMA-Oligo-Vanco. The PEGDMA-Oligo-Vanco+S. aureus group showed no significant differences in BVF, BMD, or C. Th. when compared to uninfected control groups, suggesting that the coating effectively prevented the changes in bone architecture often seen in osteomyelitis.

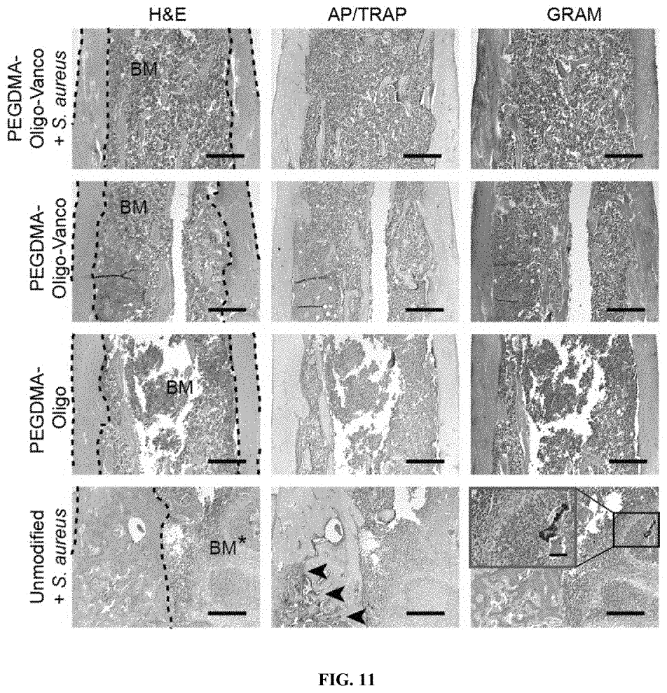

Histological staining of explanted femurs was used to corroborate the morphological changes observed by μCT and to visualize any colonization or penetration of bacteria along the endosteal surface or within the canaliculi of the cortical bone. Hematoxylin and eosin (H&E) staining revealed normal cortical bone structure and bone marrow morphology in the PEGDMA-Oligo-Vanco+S. aureus group while pronounced cortical thickening was found in the PEGDMA-Oligo control+S. aureus (FIG. 5c) and unmodified Ti6Al4V+S. aureus (FIG. 11) groups, consistent with μCT findings. A highly cellularized bone marrow canal was also detected in the infected groups treated with unmodified pins (FIG. 11, bottom row, *) or pins with the PEGDMA-Oligo control coting (FIG. 5c, middle row, *), consistent with elevated cellular responses to an active local infection. No alkaline phosphatase (AP, blue. osteoblasts) or tartrate-resistant acid phosphatase (TRAP, red, osteoclasts) activities, indicative of active bone remodeling were found in the PEGDMA-Oligo-Vanco+S. aureus group (FIG. 5c, top row) or the uninfected groups (FIG. 11, first 2 rows). By contrast, infected femurs treated with unmodified pins (FIG. 11, bottom row) or pins with the PEGDMA-Oligo control coating (FIG. 5c, middle and bottom rows) exhibited coupled osteoblastic and osteoclastic activities within the cortical bone, consistent with the observed cortical thickening and enhanced remodeling within the cortices. Gram staining revealed colonized bacteria (blue) within the bone marrow canal of the PEGDMA-Oligo+S. aureus (FIG. 5c, middle and bottom rows) and unmodified pins+S. aureus groups (FIG. 11, bottom row, inset) but not in the PEGDMA-Oligo-Vanco+S. aureus group (FIG. 5c, top row) or the control groups without S. aureus inoculation (FIG. 11, 2 middle rows). No bacteria were detected in the cortical bone canaliculi by optical microscopy.

These μCT and histological findings strongly corroborated the bioluminescence data and endpoint bacterial counts on the retrieved pins to demonstrate the effectiveness of PEGDMA-Oligo-Vanco coated Ti6Al4V pins in eradicating S. aureus within the marrow canal in a timely manner to prevent their colonization on implant surface or invasion into the cortical bone. These outcomes represent a significant improvement over the short-range suppression of bacterial colonization on Ti6Al4V IM pins achieved by vancomycin covalently conjugated to either surface oxides or to implant surface-grafted polymer brushes. (Antoci, et al. 2007 Clin Orthop Relat Res 461, 88-95; Francis, et al. 2000 Infect Immun 68 (6), 3594-600; Zhang, et al. 2019 ACS Appl Mater Interfaces. 11, 32, 28641-28647.) The on-demand release of vancomycin from the hydrogel coating enabled the bactericidal properties of the freed vancomycin be exerted in the broader periprosthetic tissue environment in a timely manner, clearing the bacteria before they had a chance to colonize on implant surfaces or invade the bone.

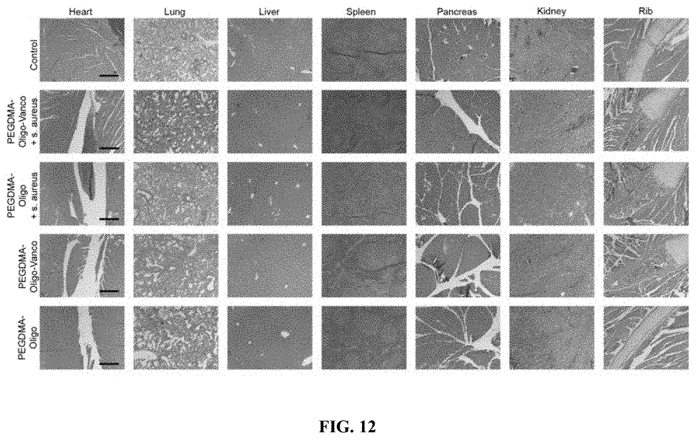

Scavenger organs harvested at 21 days post-operation revealed no difference among any of the groups treated with hydrogel-coated pins (both infected and uninfected) versus the healthy controls (FIG. 12), supporting the safety of the coatings including the subsequently released vancomycin within the timeframe examined. Such a safety profile is not surprising given that the polymethacrylate chemistry is employed in commercial bone cements and the covalent vancomycin loading dose of 0.6 μg/mg PEGDMA applied to the Ti6Al4V IM pin was 200-400 fold lower than the common prophylactic antibiotics loading doses in commercial bone cement

Taken together, these results support outstanding bactericidal properties of the PEGDMA-Oligo-Vanco coating applied to Ti6Al4V pins to protect against S. aureus infections, eradicating the bacteria from both the implant surface and its periprosthetic bony tissue environment. The low dose of 40 CFU of S. aureus inoculated was sufficient to establish infection in the untreated groups and emulates a realistic clinical setting where, following standard debridement, gross infections prior to implantation is unlikely; it supports the validity of this model for examining the efficacy of prophylactic bactericidal coating. By contrast, literature shows that in the absence of an MN-sensitive linker, antibiotics covalently attached to metallic implants can only reduce the bacterial colonization/biofilm formation on the implant surface, but are unable to release free-diffusing vancomycin to the broader periprosthetic tissue space to prevent the invasion, proliferation and colonization to tissue and ultimately the development of chronic infections.

### Tethering Oligo-Vanco to a Degradable Hydrogel Network

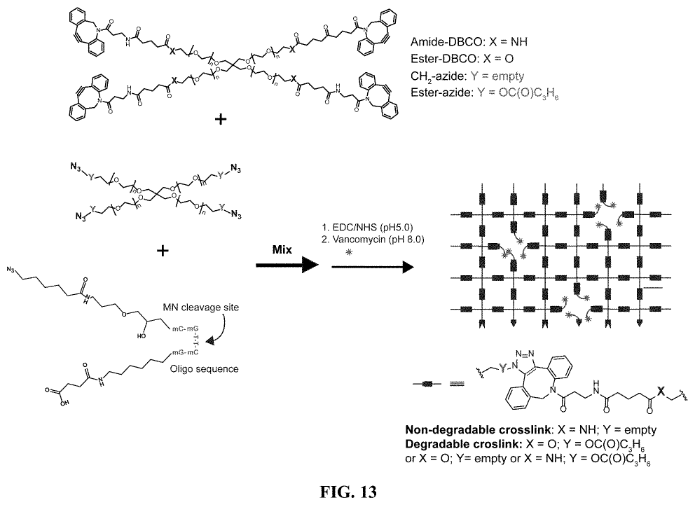

The oligonucleotide-drug construct, with various modifications on the nucleotide for enhancing its stability against non-specific cleavage, can also be covalently tethered to degradable hydrogel network. One is example is show in FIG. 13 where an azide-terminated oligonucleotide is covalently conjugated with a hydrogel network via azide-alkyne click chemistry by mixing with it with azide- and cyclooctyne-terminated macromer building blocks with various fractions of hydrolysable ester linkages.

### Fusion 2 Oligonucleotides

The oligonucleotide linker may also be the fusion of two single or double-stranded oligo sequences each sensitive to cleavage by distinct bacterial nucleases, for instance, one by Gram positive S. aureus's MN and the other by Gram negative E. coli's endonuclease I, a predominant DNase in E. coli (Flenker, et al 2017 Mol Therapy, 25 (6), 1353-1362).

Modified S. aureus Sensitive Oligonucleotide Sequences

The following 5′ and 3′ modified S. aureus sensitive Oligo sequences were synthesized (m denotes methylation while * denotes phosphorothioate/PS modification):
1. /5Carboxyl/mCmGTTmCmG/3Acryd/(OPS)
2. /5Carboxyl/*mCmGTTmCmG*/3Acryd/(2PS-a)
3. /5Carboxyl/mCmG* TTmC*mG/3Acryd/(2PS-b)
4. /5Carboxyl/*mC*mGTTmC*mG*/3Acryd/(4PS)
5. /5Carboxyl/*mC*mG*TT*mC*mG*/3Acryd/(6PS)
6. /5Carboxyl/*mC*mG* TTmC*mG*/3Acryd/(5PS)

The following 5′ and 3′ modified E. coli sensitive Oligo (duplex) sequences were synthesized:
Sequence.1: /5Carboxyl/CTACGTAG/3Acryd/; Sequence-.2: /5Carboxyl/CTACGTAG/3Acryd/
   Digestion of Oligos with Varying Numbers of PS Modifications by MN

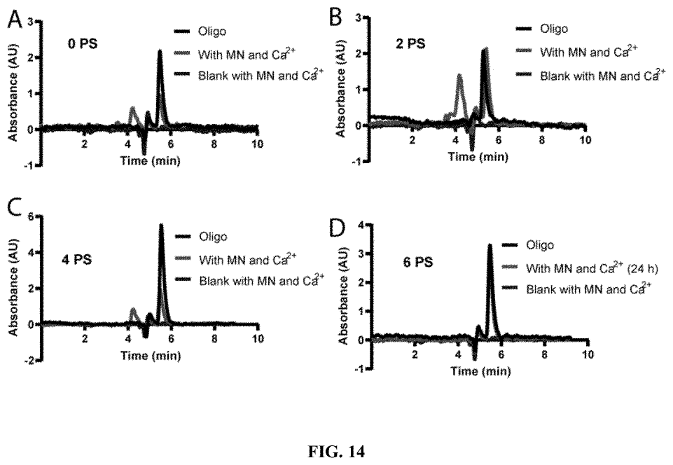

The digestion of the phosphorothioate (PS)-modified oligos by micrococcal nuclease (MN, 0.1-1.0 U/mL) was monitored by aqueous gel permeation chromatography (GPC) equipped with a C18 columns and a UV detector. As shown in FIG. 14, the stability of PS-modified Oligo towards MN cleavage increased, with the extent of stability enhancement positively correlating with the number of PS modifications. The location of PS modifications also has an impact on the stability. These data support that both methylation and PS modifications can be strategically utilized, either alone or in combination to fine-tune the stability and cleavage kinetics for varying applications.

S. aureus Triggered Release of Antibiotics from the PEGDMA-Oligo-Gentamicin and PEGDMA-Oligo-Tobramycin Hydrogels

20,000 CFU of S. aureus suspension was spread evenly on an LB agar plate and incubated for 10 min at 37° C. with shaking. Hydrogel discs of PEGDMA-Oligo-Gentamicin, PEGDMA-Oligo-Tobramycin, PEGDMA-Oligo/Gentamicin control (gentamicin was physically encapsulated during hydrogel formation but subsequently washed away) or PEGDMA-Oligo/Tobramycin control (tobramycin was physically encapsulated during hydrogel formation but subsequently washed away) were then placed over the LB agar plate and the culture was continued for 24 h at 37° C. with shaking. The clear zones surrounding the hydrogels were photo-documented as an indication of antibiotic activities surrounding the hydrogel discs. PEGDMA-Oligo hydrogels with physically encapsulated but subsequently washed away antibiotics served as negative controls.

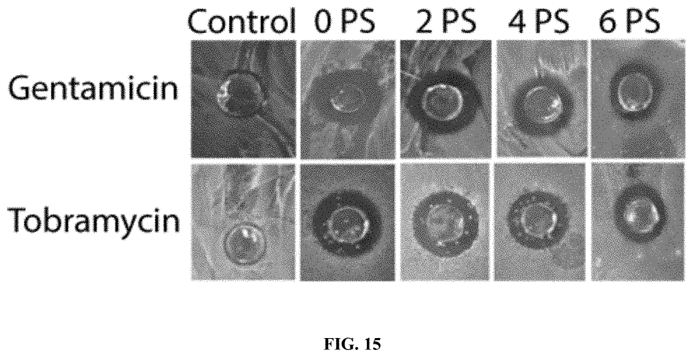

As shown in FIG. 15, the cleavage sensitivity of the broad-spectrum antibiotics from the PEGDMA-Oligo-Gentamicin and PEGDMA-Oligo-Tobramycin hydrogels in response to the presence of the S. aureus inversely correlated with the degree of PS modifications. The dimension of the clear zones developed around the hydrogel discs placed on S. aureus agar plates, reflecting the bactericidal effect of the cleaved/released free gentamicin or tobramycin, inversely correlated with the number of PS modification of the respective oligo linker between the tethered antibiotics and the crosslinked hydrogel.

### Specific and Nonspecific Cleavage of PS-Modified Oligos by MN and Human Serum:

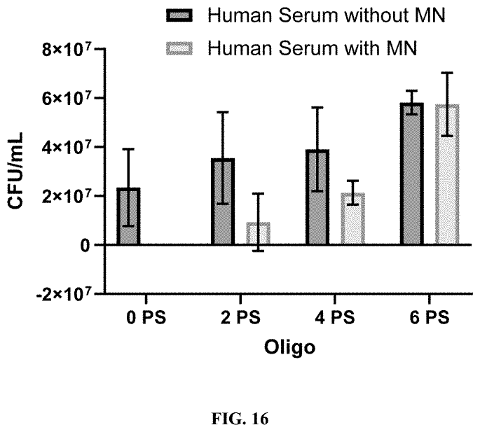

Specific cleavage by MN and nonspecific cleavage by other nucleases present in human serum was studied by incubating the PEGDMA-Oligo-Gentamicin hydrogel in human serum (with and without MN) for 2 h and examining how the supernatants obtained after removing the hydrogel differentially inhibited S. aureus culture. As shown in FIG. 16, serum/MN supernatant obtained after 2-h incubation with the hydrogel containing 0 PS oligo showed least or no CFU count, indicating that the oligo was susceptible to both specific and nonspecific cleavages by MN and other nucleases in human serum, thereby releasing cleaved bactericidal gentamicin. However, with an increase in the number of PS modifications in the oligo linker (e.g. 2 PS, 4 PS, and 6 PS), the total CFU counts increased accordingly, indicating the reduced oligo cleavage kinetics in both specific and nonspecific cleavages. The data also support faster oligo cleavage by MN than by other nucleases present in the human serum.

### Experimental

### Materials

All chemicals were purchased from Sigma-Aldrich unless specified. For water contact angle and XPS characterizations, Ti6Al4V plates (0.5-mm thick, TMS Titanium, Poway, Calif.) were cut into 1×1 cm² square pieces and subjected to surface modifications. For in vivo studies, Ti6Al4V wires (0.5 mm in diameter, Goodfellow Corporation, Coraopolis, Pa.) were cut into 1-cm long pins for surface modifications. The methacrylated oligonucleotides were synthesized by IDTDNA (Coralville, Iowa) and used as received. Staphylococcus aureus (S. aureus, ATCC 25923) was purchased from ATCC and bioluminescent S. aureus strain Xen29 was purchased from Perkin Elmer.

### Enzymatic Oligo Digestion Monitoring

The digestion of the methacrylated oligo by micrococcal nuclease (MN, 0.1-1.0 U/μL) was monitored by aqueous gel permeation chromatography (GPC) on a Varian ProStar HPLC system with two PL Aquagel-OH columns (Agilent Technologies) and a UV detector. The eluent was aqueous PBS (10 mM, pH 7.4) and the flow rate was 1.0 mL/min. Detection of the oligo probe and its fragments upon digestion was enabled by UV absorbance at 260 nm. Extinction coefficient of 2′-O-methyl-modified nucleotides was assumed to be the same as that of RNA. All measurements were performed in triplicates.

For the digestion of the phosphorothioate (PS)-modified oligos by MN (0.1-1.0 U/mL) C18 columns (Agilent Technologies) was used and was monitored via UV detector. The eluent was 10% (v/v) acetonitrile containing 0.1% (v/v) trifluroacetic acid and the flow rate was 0.75 mL/min. Detection of the oligo probe and its digested fragments upon digestion was enabled by UV absorbance at 260 nm.

### Synthesis of PEGDMA-Oligo and PEGDMA-Oligo-Vanco Hydrogels

To synthesize the PEGDMA-Oligo hydrogel with varying crosslinking densities, a hydrogel precursor solution containing PEGDMA (7.5, 10 or 15 wt %), the methacrylated oligo (75 μM) and VA-086 (initiator; 1 wt %) in deionized (DI) water was freshly prepared. For the hydrogel disc preparation, 50 μL of the precursor solution was photo-crosslinked in an 8-mm in diameter Teflon mold under 365-nm irradiation for 10 min. All hydrogels were washed in DI water for 72 h with frequent fresh water changes to remove excess radical initiators or any untethered methacrylate prior to further experiments.

Vancomycin was covalently attached to PEGDMA-Oligo hydrogel via EDC/NHS coupling. First the PEGDMA-Oligo hydrogel was equilibrated in 900-μL PBS (10 mM, pH 5.0) for 60 min. Then, EDC and NHS (7.5 mM, 50 μL each) were added to the equilibrated hydrogel and allowed to react for 30 min in RT. The activated hydrogel was then transferred into a vancomycin solution (0.5 mL, 150 μM in 10 mM PBS, pH 8.0) for amidation at RT for 4 h. All covalently modified PEGDMA-Oligo-Vanco hydrogels were washed in DI water for 72 h with frequent fresh water changes to remove unreacted/excess EDC, NHS and/or vancomycin prior to further use.

The oligonucleotide coupling efficiency within the hydrogel was determined by a leaching experiment. Briefly, the PEGDMA-Oligo-Vanco hydrogel (50 μL, 8 mm diameter) was immersed in DI water (0.5 mL) and the absorbance of the supernatant collected every hour for six hours and once after 24 and 48 hours was recorded at 260 nm. The accumulative vancomycin collected from the supernatant after 48 h was considered not covalently coupled to the hydrogel. By this experiment, it was determined that a 90% coupling efficiency of vancomycin was achieved on the 15 w/v % PEGDMA hydrogel, which is considered adequate and applied to all subsequent experiments.

### Synthesis of Gentamycin and Tobramycin-Tethered Oligo and the Preparation of PEGDMA-Oligo-Gentamicin and PEGDMA-Oligo-Tobramycin Hydrogels

Also studied was the feasibility of covalently conjugating broad-spectrum antibiotics such as gentamicin and tobramycin to the oligo. Briefly, gentamycin and tobramycin were covalently attached to the/5Carboxyl/-Oligo-/3Acryd/ via EDC/NHS coupling. First, the 5′-carboxylic acid endgroup of the Oligo was activated by adding EDC and NHS ([COOH]:[EDC]:[NHS]=1:1:2) for 10 min in 10 mM PBS (pH 6.0). Antibiotics (Gentamicin or Tobramycin) was then added to the activated Oligo in the ratio of [COOH]:[Antibiotics]=1:2 and stirred at rt for 4 hours to obtain 5′-gentamicin or 5′-tobromycin-tethered Oligo.

For the hydrogel disc preparation, 50 μL of the 5′-gentamicin or 5′-tobromycin-tethered Oligo (with 3′-Acrydite) precursor solution containing PEGDMA (15% v/v) and VA-086 (initiator; 1 wt %) was photo-crosslinked in a Teflon mold (an 8-mm in diameter) under 365-nm irradiation for 10 min. All crosslinked hydrogels were washed in DI water for 72 h with frequent fresh water changes to remove residue radical initiators or any untethered methacrylate prior to further experiments.

In Vitro Release Kinetics of Vancomycin from Modified Hydrogels

The PEGDMA-Oligo-Vanco hydrogels (50 μL, 8 mm diameter) were immersed in 0.5 mL of sterile PBS (10 mM, pH 7.4) containing 0.1 U/μL of MN and incubated at 37° C. At predetermined time points (1, 1.5, 2, 3, 4, 6, 12, 16, 24 h), the amount of vancomycin released from the hydrogel matrix was calculated from the absorbance measured with the UV spectrophotometer at 280 nm. All the measurements were performed in triplicates.

### Specific and Nonspecific Cleavage of PS-Modified Oligos by MN and Human Serum:

Specific cleavage by MN and nonspecific cleavage by other nucleases present in human serum was studied by incubating the PEGDMA-Oligo-Gentamicin hydrogel in human serum (with and without MN) for 2 h and examining how the supernatants obtained after removing the hydrogel differentially inhibited S. aureus culture. Briefly, the PEGDMA-Oligo-Gentamicin hydrogel (50 μL, 8 mm in diameter, n=3) was immersed in 0.25 mL of human serum with or without MN (0.1 U/mL) and incubated at 37° C. for 2 h. The hydrogel was then removed while 0.25 mL of LB media containing 250 CFU S. aureus was added. The culture of S. aureus was incubated for 24 h at 37° C. with shaking. Total bacterial CFU counts were determined at 24 h by sample turbidity (OD₆₀₀), measured using UV-Vis spectroscopy at 600 nm and calculated using a standard curve.

### Hydrogel Coating of Ti6Al4V Pins

To enhance the adhesion and stability of the hydrogel coating, Ti6Al4V pin surfaces were first modified using dopamine methacrylate (DopaMA). Ti6Al4V pins were immersed into 0.5 mL of ethanol containing 1 mg/mL of dopamine methacrylate for 12 h at RT. The pins were then rinsed with ethanol 5 times to remove excess of dopamine methacrylate and dried under vacuum for 4 h. The DopaMA-coated pins were immersed into freshly prepared hydrogel precursor solution containing 15 wt % PEGDMA, 75 μM of methacrylated oligo and 1 wt % of VA-086. It was then partially photo crosslinked under 365 nm irradiation for 2 min. Pins are then removed from the pre-gel condition and then irradiated for 5 min to complete the crosslinking. All hydrogels coated pins were washed in DI water for 72 h with frequent fresh water changes to remove excess radical initiators or any untethered methacrylate prior to further experiments.

In Vitro Antibacterial Activity of Ti6Al4V Pins Coated with PEGDMA-Oligo-Vanco Hydrogel or PEGDMA-Oligo Hydrogel with/without Physically Encapsulated Vancomycin. The therapeutic efficacy of the PEGDMA-Oligo-Vanco hydrogel was evaluated by its ability to inhibit in vitro suspension culture of S. aureus (method I) or its ability to inhibit S. aureus growth on agar plate when delivered in the form of Ti6Al4V coating (method II). PEGDMA-Oligo hydrogel without covalent vancomycin tethering or that with physically encapsulated but subsequently washed away vancomycin served as negative controls. Method I. PEGDMA-Oligo-Vanco, PEGDMA-Oligo or PEGDMA-Oligo/Vanco (after washing away physically encapsulated vancomycin) hydrogels (50 μL, 8 mm diameter) were immersed in 0.5 mL of LB media containing 130 CFU of S. aureus. Cultures were incubated for 48 h at 37° C. with shaking. Total CFU counts were determined at 24 and 48 h by sample turbidity (OD₆₀₀), measured using UV-Vis spectroscopy at 600 nm and calculated based on standard curves. Method II. 20,000 CFU of S. aureus were spread evenly on LB agar plates and incubated for 10 min at 37° C. with shaking. Ti6Al4V pins coated with PEGDMA-Oligo-Vanco, PEGDMA-Oligo or PEGDMA-Oligo/Vanco (after washing away physically encapsulated vancomycin) were then placed on the plates and the culture was continued for 24 h at 37° C. with shaking. The clear zones surrounding the pins were photo-documented as an indication of local antibiotic activities as a function of pin coatings.

### Water Contact Angle Measurement

For the water contact angle measurements, Ti6Al4V plates (10 mm×10 mm) were used. The static water contact angles of Ti6Al4V substrates before and after the surface coating by dopamine methacrylate was recorded on a CAM200 goniometer (KSV Instruments). A droplet (3 μL) of Milli-Q water was placed on the substrate and the contact angles (left and right) of the droplet were recorded after 30 s. The left and right contact angles of each droplet, and three substrates of each sample group were averaged and reported as averages±standard deviation.

### X-Ray Photoelectron Spectroscopy (XPS)

Surface compositional analyses of Ti6Al4V and Ti6Al4V-DopaMA substrates were carried out on a Thermo Scientific K-Alpha XPS equipped with an Al K_{α} radiation source under the pass energy of 200 or 50 eV (for survey or high-resolution scan) and the spot size of 400 μm. Survey scan spectra were obtained from five consecutive scans of a randomly chosen area of interest.

### In Vivo Studies

All animal procedures were carried out per procedures approved by the University of Massachusetts Medical School Institutional Animal Care and Use Committee (IACUC). CL57BL/6 male mice ages 6-10 weeks and weights 22-28 g were anesthetized with 2-3% isoflurane-oxygen throughout surgery and bilateral intramedullary pins were inserted following bacteria inoculation. Briefly, a 2 mm skin incision was made over the knee exposing the joint. A medial incision was made to dislocate the patellar tendon, and the patella and patellar tendon were moved laterally to reveal the intercondylar notch of the femur. A 25-gauge needle was introduced onto the patellar grove and used to reamed through the femur. 4 μL of sterile LB media or Xen29 S. aureus solution (10⁴ CFU/mL) was injected into the femoral canal (0 or 40 CFU Xen29 per femur) using a micro pipettor equipped with a 25-gauge needle tip. A sterile unmodified Ti6Al4V rod (0.5 mm in diameter and 10 mm in length) or those coated with PEGDMA-Oligo-Vanco or PEGDMA-Oligo hydrogels via a dopamine intermediate coating was fit into the previously reamed femur. The patella and patellar tendon were placed back into position and the skin was closed with 5-0 nylon sutures. Subcutaneous injections of buprenorphine were given immediately pre-operation and every 12 h post-operation for 48 h.

### Micro Computed Tomography (μCT)

Mice were scanned 1 day (to ensure proper pin placement and normal femoral anatomy) and 21 days post-operation on a Scanco vivaCT 75 system (Scano Medical, Switzerland) at an effective voxel size of 20.5×20.5×20.5 μm³. The proximal and distal femoral growth plates were located to establish the center slice of the femur and 100 consecutive slices were analyzed on both sides for a total analysis length of ˜4 mm. A global threshold of 260 (minimum bone densities of 549.7 mg HA/cm³ and above) was applied to calculate bone volume fraction (BVF) and bone mineral density (BMD) using Scanco Medical's analysis software. When analyzing cortical thickness, a threshold of 50 (minimum bone densities of 63.2 mg HA/cm³ and above) was utilized to include the entire contoured cortical bone space despite any porosity or lesions within the cortical bone.

### In Vivo Bioluminescence Imaging

The bioluminescence imaging of S. aureus within the infected mouse femoral canals was carried out using IVIS-100 imaging system (Perkin-Elmer) on day 2, day 7, day 14 and day 21 post-op. Mice were anesthetized with 5% isoflurane-oxygen and placed on the imaging platform. The bioluminescence image was recorded with a 5 min overall exposure time with open emission filter.

### Explant Bacterial Counts

At 21 days post infection, mice were euthanized and the IM pins were retrieved. Each retrieved pin was placed in 0.5 mL of LB media in an eppendorf tube and vortexed for 5 min to dislodge all surface-bound bacteria from the pin. The CFU counts were determined by serial dilution on LB agar plates.

### Femoral Histology and Organ Pathology

At 21 days, immediately after IM pin removal, femoral explants were fixed in periodate-lysine-paraformaldehyde (PLP) for 48 h at 4° C. followed by decalcification in 18% aqueous ethylenediaminetetraacetic acid (EDTA, pH 8.0) for 2 weeks with bi-weekly solution changes. (Kutikov, et al. 2015 ACS Appl Mater Interfaces 7 (8), 4890-901.) The decalcified femurs were subjected to serial dehydration, paraffin embedding, and longitudinal sectioning (6 μm in thickness) before staining for hematoxylin and eosin (H&E) for cellularity, osteogenic marker alkaline phosphatase (AP, fast blue) and osteoclast lineage marker tartrate-resistant acid phosphatase (TRAP, fast red) for bone remodeling, and by gram stain kit (Abcam) for gram positive S. aureus (blue). Heart, lung, kidney, liver, pancreas, spleen, and ribs were collected at 21 days post-operation and fixed and stained by H&E for comparison with organs retrieved from healthy age-matched controls.

### Statistics and Study Design

All statistical analysis was performed using Prism 7.0 (GraphPad Software Inc.) Shaprio-Wilk normality testing was used to evaluate data distribution. Pair-wise comparisons passing normality test were analyzed with Student's t-test while the Mann-Whitney rank-sum test was used for pair-wise comparison of non-parametric data. Multiple group comparisons passing normality test were analyzed using one-way analysis of variance (ANOVA) with Tukey's posthoc while non-parametric multiple group comparisons were analyzed using the Kruskal-Wallis rank-sum test with Dunn's posthoc testing. Multi-variant comparisons were carried out using two-way ANOVA with Tukey's posthoc test. P-values of <0.05 were considered significant. All data was presented as mean±standard deviation (S.D.).

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description, herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. A polymer-drug conjugate comprising an oligonucleotide of about 2 to about 30 nucleotides in length having a first end and a second end, wherein the oligonucleotide is linked at the first end to a polymer and at the second end to an antimicrobial agent.

2. The polymer-drug conjugate of claim 1, wherein the oligonucleotide is single stranded.

3. The polymer-drug conjugate of claim 1, wherein the oligonucleotide is double stranded.

4. The polymer-drug conjugate of claim 1, wherein the oligonucleotide is a fusion of one or more single or double stranded oligonucleotides sensitive to cleavage by one or more bacterial nucleases.

5. The polymer-drug conjugate of claim 1, wherein the oligonucleotide is adapted to be selectively cleaved by a first microbial nuclease.

6. The polymer-drug conjugate of claim 1, wherein the first microbial nuclease is micrococcal nucleases (MN) of S. aureus.

7. The polymer-drug conjugate of claim 1, wherein the oligonucleotide is adapted to be cleaved by a second or further microbial nucleases.

8. The polymer-drug conjugate of claim 1, wherein the oligonucleotide is substantially more stable against cleavage by a mammalian nuclease than against the first, second or further microbial nucleases.

9. The polymer-drug conjugate of claim 1, wherein the oligonucleotide is about 2 to about 20 nucleotides in length.

10. The polymer-drug conjugate of claim 1, wherein the oligonucleotide comprises one or more chemically modified pyrimidines and purines.

11. The polymer-drug conjugate of claim 1, wherein the one or more chemical modifications comprise one or more 2′-O-carboxymethyl or 2′-fluoro modifications.

12. The polymer-drug conjugate of claim 1, wherein the one or more chemical modifications comprise one or more of phosphorothioate modifications.

13. The polymer-drug conjugate of claim 1, wherein the oligonucleotide comprises a sequence selected from the group consisting of mC-mG-T-T-mC-mG, C*-G*-T-T-C*-G*, mC*-mG*-T-T-mC*-mG*, mC-mG*-T-T-mC*-mG, mC*-mG-T-T-mC-mG*, and mC-mU-mC-mG-T-T-mC-mU-mC-mG, wherein m denotes 2′-O-methylation while * denotes phosphorothioate modification.

14. The polymer-drug conjugate of claim 1, wherein a first spacer is present between the oligonucleotide and the polymer and/or a second spacer between the oligonucleotide and the antimicrobial agent.

15-24. (canceled)

25. A hydrogel composition comprising a polymer-drug conjugate of claim 1.

26. A coating, surface or surface layer comprising a polymer-drug conjugate of claim 1.

27. A nanoparticle formulation comprising a polymer-drug conjugate or a hydrogel composition of claim 1.

28. A medical device or implant comprising a polymer-drug conjugate of claim 1.

29. A medical device or implant comprising a coating, surface or a surface layer of a hydrogel composition, wherein the hydrogel composition comprises an oligonucleotide of about 2 to about 30 nucleotides in length having a first end and a second end, wherein the oligonucleotide is linked at the first end to a polymeric network and at the second end to an antimicrobial agent, wherein the oligonucleotide is adapted to be selectively cleaved by a first microbial nuclease and not cleaved by a mammalian nuclease.

30-48. (canceled)

48. A compound having the structural formula: 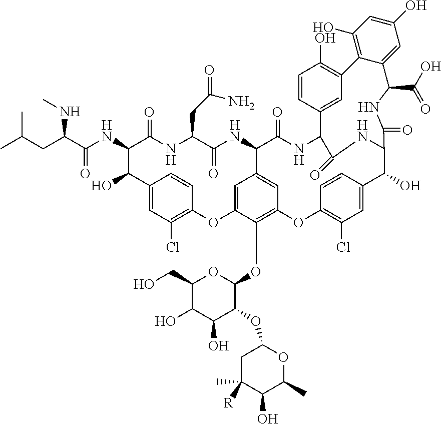
wherein R comprises an oligonucleotide having about 1 to about 29 nucleotide units.

49-75. (canceled)
